(19) United States
(12) Patent Application Publication
(10) Pub. No.: US 2009/0149375 A1
(43) Pub. Date: Jun. 11, 2009

(54) USE OF THE MST PROTEIN FOR THE TREATMENT OF A THROMBOEMBOLIC DISORDER

(75) Inventors: Paola Concari, Frankfurt am Main (DE); Matthias Herrmann, Frankfurt am Main (DE); Jochen Kruip, Frankfurt am Main (DE); Thomas Leeuw, Frankfurt am Main (DE); Alexander Rueck, Buchs (CH)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 11/908,467

(22) PCT Filed: Mar. 20, 2006
(86) PCT No.: PCT/EP2006/002547

§ 371 Date: Jul. 9, 2008

(30) Foreign Application Priority Data
Mar. 23, 2005 (EP) 05006359.3

(51) Int. Cl. A61K 38/16; A61K 31/7088; C12Q 1/68; C12Q 1/48; C40B 30/00

(52) U.S. Cl. 514/12; 514/44; 435/6; 435/15; 506/7

(57) ABSTRACT

The present invention refers to the use of the Mst protein or a nucleotide sequence coding for the Mst protein for the treatment of a thromboembolic disorder and to a method of screening a modulator of the Mst protein or the nucleotide sequence coding for the Mst protein.

The present invention refers to the use of the Mst protein or a nucleotide sequence coding for the Mst protein for the treatment of a thromboembolic disorder and to a method of screening a modulator of the Mst protein or the nucleotide sequence coding for the Mst protein.

During recent years, several antiplatelet therapies, ranging from aspirin to ticlopidine and clopidrogel, have been introduced and their benefits are well documented. However, although all these different therapies add additional benefits for the treatment of thromboembolic disorders, they are often associated with non-desirable side effects and the efficacy of even combined treatments is still not sufficient. Thus, novel approaches for the treatment of thromboembolic disorders are urgently needed.

The STE20-related kinases constitute an evolutionarily conserved family of serine/threonine kinases. Step 20p, the founder of this kinase family, is a MEK kinase kinase kinase (MAP4K) involved in the pheromone response pathway of budding yeast (Leberer, E. et al. (1992) EMBO J. 11, 4815-4824). In recent years a number of mammalian and yeast homologs of Step 20 have been identified. They fall into two classes: those binding Cdc42 and/or Rac1 (p21-activated kinases or PAKs), and those that do not appear to be regulated in this manner (germinal center kinases or GCKs) (for review see Dan, C. et al. (2001) J. Biol. Chem. 276, 32115-32121). Mst1 is a member of the latter class: it has been shown to be homologous to the yeast Step 20 and mammalian Pak enzymes throughout the kinase domain, but does not contain the p21 GTPase-binding domain, nor does it share any significant homology with Step 20 or Pak outside the kinase domain.

The human Mst1 (Mammalian Sterile Twenty-like) has been originally isolated by PGR screening of a human lymphocyte cDNA library with degenerate primers designed to amplify the catalytic domains of serine/threonine kinases (Creasy, C. L. et al. (1995) J. Biol. Chem. 270, 21695-21700; Creasy, C. L. et al. (1995) Gene 167, 303-306). A close homolog of Mst1, Mst2 was identified shortly thereafter by the same authors. In one early study both, Mst1 and Mst2 have been shown to be activated in response to stress conditions and apoptotic agents and have been therefore alternatively named Kinase Responsive to Stress (Krs) 1 and 2.

Recent publications suggest a role for Mst1 and Mst2 in apoptosis. Both Mst1 and Mst2 undergo caspase-mediated proteolysis in response to apoptotic stimuli, such as ligation of CD95/Fas or treatment with staurosporine (Graves, J. D. et al. (1998) EMBO J 17, 2224-2234; Lee, K. K. et al. (1998) Oncogene 16, 3029-3037). However, while Mst1 has two different caspase-cleavable sites, which generate two biochemically distinct catalytic fragments, just one caspase-cleavable site has been reported for Mst2. Due to the negative regulatory effect of the C-terminal domains of Mst1 and Mst2, their removal by caspase-cleavage activates the kinase activity of the resulting forms in vitro and in vivo, and causes cellular translocation (Creasy, C. L. et al. (1996) J. Biol. Chem. 271, 21049-21053; Deng, Y. et al. (2003) J. Biol. Chem. 278, 11760-11767; Graves, J. D. et al. (2001) J. Biol. Chem. 276, 14909-14915; Graves, J. D. et al. (1998) supra; Lee, K. K. et al. (1998) supra). Furthermore, overexpression of Mst1 induces morphological changes characteristic of apoptosis in human B lymphoma cells (Graves, J. D. et al. (2001), supra. cDNA cloning of MST homologues in mouse and nematode shows that caspase-cleaved sequences are evolutionarily conserved.

Overexpression of Mst1 activates the JNK and p38 MAP kinase pathways via MKK4/MKK7 and MKK3/MKK6, respectively (Graves, J. D. et al. (2001) supra; Ura, S. et al. (2001) Genes Cells 6, 519-530). As expression of Mst1 results in caspase-3 activation, Mst1 is not only a target of caspases but also an activator of caspases. This caspase activation and apoptotic changes occur through JNK, since the co-expression of a dominant-negative mutant of JNK inhibited Mst1-induced morphological changes as well as caspase activation (Ura, S. et al. (2001) supra). In a recent paper, (Khokhlatchev, A. et al. (2002) Curr. Biol. 12, 253-265 strong support for the existence of a novel Ras effector pathway influencing cell survival has been shown. Following this model, activated Ras can bind to a complex consisting of Nore and Mst1 and subsequently effect downstream signaling pathways (Khokhlatchev, A. et al. (2002) supra). However, potential physiological direct substrates of Mst1 have not been identified until now. Mst1 is expressed in megakaryocytes, the progenitor cells for platelets (Sun, S. et al. (1999) J. Cell Biochem. 76, 44-60). Megakaryocytes undergo endomitotic cell cycles as part of their maturation process. In addition to polyploidization, a set of genes such as platelet factor 4 (PF4), acetylcholine esterase, and glyco-protein IIb (GPIIb) are turned on in maturing megakaryocytes. Megakaryocyte differentiation is promoted by the cytokine thrombopoietin (TPO) as the c-MpI receptor ligand. TPO signals to DNA level regulation via the Janus kinase family members JAK2 and Tyk2, She, the Stat-proteins 3 and 5, and via extracellular signal-regulated kinase 2. Mst1 expression and Mst1 kinase activity are upregulated by MpI ligand in cultured bone marrow cells and in the mouse megakaryocytic cell line Y10/L8057. In addition, the induced expression of Mst1 enhanced the expression of various differentiation markers and increased polyploidization in response to PMA (Sun, S. et al. (1999) supra).

It has been recently reported that Mst2 participates to Raf-1 signaling pathway: upon serum starvation Mst2 co-precipitates with Raf-1 in COS-1 cells transfected with a Flag-tagged Raf-1 as well as in untransfected cells (O'Neill, E. et al. (2004) Science 306, 2267-2270). Mst2 is a kinase whose activity is increased by pro-apoptotic agents via homodimerization and transphosphorylation (Deng, Y. et al. (2003) supra; Lee, K. K. et al. (1998) supra). O'Neill and colleagues have shown that both these processes are inhibited by Raf-1 through a mechanism that is not dependent on Raf-1 kinase activity, but probably relies on the recruitment of a still unidentified Mst2 phosphatase (O'Neill, E. et al. (2004) supra).

By means of proteomics approaches we found that Mst1 and Mst2 are expressed in human platelets. Meanwhile these findings have been confirmed by other investigators: by using classical 2D-gel based proteomics analysis of platelet cell extracts, O'Neill et al. have confirmed the expression of Mst2 in platelets (O'Neill, E. (2002) Proteomics 2, 288-305). In addition, Kris Gevaert and collaborators have identified Mst1 and Mst2 phosphoproteins in platelets (Gevaert, K. et al. “Novel Strategies and Applications for Non-gel Proteomics”, Proteomic Forum München 2003—International Meeting on Proteome Analysis, Sep. 14-17, 2003, Munich, Germany) using a Combined Fractional Diagonal Chromatography (Gevaert, K. et al. (2003) Nat. Biotechnol. 21, 566-569). However, although identifying Mst1 and/or Mst2 in platelets, none of these studies provided any data about a potential function for Mst kinases in platelets. Thus, up to now, no potential role for Mst family kinases in platelet signaling and platelet activation has been described.

Now, the present invention refers to the finding that the Mst protein is involved in signal transduction events finally leading to platelet activation and aggregation.

Consequently, the present invention is directed to the use of the Mst protein, in particular of Mst 1 and/or Mst 2, or a nucleotide sequence coding for the Mst protein, in particular for Mst 1 or Mst 2, for the treatment of a thromboembolic disorder, in particular for the production of a medicament for the treatment of a thromboembolic disorder. Preferably the Mst protein, in particular the Mst 1 and/or Mst 2 protein, is a human Mst protein. It is noted that in general human Mst1 and human Mst2 have an identity of 77.6% at the amino acid level. The Zebrafish genome contains only one homolog representing both, Mst1 and Mst2 with an amino acid identity of 76.8% and 88.8%, respectively. The human and the rat MST2 sequences have a percent of identity of 96.7% at the amino acid level.

In a preferred embodiment the human Mst 1 protein or its nucleotide sequence is characterized by the amino acid sequence of SEQ ID NO: 1 or the nucleotide sequence SEQ ID NO: 2, respectively, and the human Mst 2 protein or its nucleotide sequence is characterized by the amino acid sequence of SEQ ID NO: 3 or the nucleotide sequence SEQ ID NO: 4, respectively.

According to the present invention the term Mst protein refers generally to any naturally occurring Mst protein as well as biologically active Mst mutants. Naturally occurring Mst proteins include Mst proteins of different species, e.g. of zebrafish, preferably vertebrates, more preferably mammals, as well as biologically active splice variants. The most preferred Mst proteins are already mentioned above.

The term “biologically active Mst mutant” refers to a protein derived from the Mst protein and which retains its biological activity, i.e. the kinase activity. Therefore, in the present case the term “biologically active” refers to the Mst kinase activity which can be measured in any generally known kinase assay, such as the assays described in the present specification, in particular the ATP consumption assay described in the Examples. In short, the ATP consumption assay refers to an in vitro kinase assay containing a kinase buffer solution with ATP and Mg²⁺-Ions or Mn²⁺-Ions and the kinase, here the Mst mutant, to be tested. The ATP consumption and, therefore, the kinase activity are generally measured by bioluminescence.

More particularly the term “biologically active Mst mutant” refers to an amino acid sequence which differs from the naturally occurring Mst sequence in one or more amino acids but which retains its kinase activity. Such a mutant differs from the wild-type polypeptide in the substitution, insertion or deletion of one or more amino acids. Preferred are semi-conservative, more preferred conservative amino acid substitutions. Typical substitutions are among the aliphatic amino acids, among the amino acids having aliphatic hydroxyl side chain, among the amino acids having acidic residues, among the amide derivatives, among the amino acids with basic residues, or the amino acids having aromatic residues. Typical semi-conservative and conservative substitutions are:

| Amino acid | Conservative substitution | Semi-conservative substitution |
|---|---|---|
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

In addition, changing from A, F, H, I, L, M, P, V, W or Y to C is semi-conservative if the new cysteine remains as a free thiol. Furthermore, the skilled person will appreciate that glycines at sterically demanding positions should not be substituted and that P should not be introduced into parts of the protein which have an alpha-helical or a beta-sheet structure.

The mutant generally differs in primary structure (amino acid sequence), but may or may not differ significantly in secondary or tertiary structure or in its function (kinase activity) relative to the naturally occurring protein. In any case the mutant shows an identity to the wild-type Mst 1 protein or Mst 2 protein of at least 70%, preferably at least 75%, more preferably at least 85%, even more preferably at least 95% and most preferably at least 99%.

Examples of Biologically Active Mst1 Mutants with Point Mutations are:

D326N which is resistant to protease, i.e. caspase, cleavage in DEMD³²⁶S, D349E which is resistant to protease cleavage in TMTD³⁴⁹G and D326N-D349E which is a double protease-resistant form (Graves J. D. et al. (2001), supra).

T183E, T175E, T175A, T177E and T177A which are mutations in the MST activation loop as well as D326N, S327A and S327E (Glantschnig, H. et al. (2202) J. Biol. Chem., 277, 42987-42996).

T175A and T177A which are also mutations in the MST activation loop, L444P which is a dimer-deficient variant as well as T120A and the double mutant S438A-T440A (Praskova, M. et al. (2004) Biochem. J., 381, 453-462).

Examples of Biologically Active Mst2 Mutants with Point Mutations are:

T117A and T384A (Deng, Y. et al. (2003), supra).

The mutant can also be a portion of the Mst protein sufficient for its kinase activity. The portion comprises at least 30 amino acids, preferably at least 100 amino acids, more preferably at least 300 amino acids, even more preferably at least 450 amino acids, and most preferably at least 482 amino acids for Mst1 and at least 486 amino acids for Mst2. This portion of the analogue can differ from the wild-type polypeptide portion in the substitution, insertion or deletion of one or more amino acids as detailed above. In one embodiment the Mst protein or the biologically active Mst mutant can be fused to another molecule, e.g. a protein and/or a marker, e.g. Glutathione-S-Transferase (GST).

### Examples of Biologically Active Mst1 Fragments are:

Δ327-487 and Δ350-487 which are the two catalytically active caspase cleavage products of Mst1 (Graves, J. D. et al. (2001), supra; Lee, K. K. et al. (2001) J. Biol. Chem. 276, 19276-19285; Glantschnig, H. et al. (2002), supra) and the truncated Mst1 forms amino acids 1-455, 1-430, 1-360 and 1-330, the deletions Δ331-360 and Δ331-394 (Creasy, C. L. et al. (1996), supra) as well as the Mst1 kinase domain as shown in FIG. 12.

### Example of an Biologically Active Mst2 Fragment:

Δ323-491 which is the catalytically active caspase cleavage product of Mst2 (Deng, Y. et al. (2003), supra).

In addition, the present invention is directed to the use of the Mst protein, in particular of Mst 1 and/or Mst 2, or a nucleotide sequence coding for the Mst protein for the discovery of a Mst protein modulator, in particular an inhibitor, as a medicament for the treatment of a thromboembolic disorder. Preferably the Mst protein, in particular the Mst 1 and/or Mst 2 protein, is a human Mst protein, also as specified above.

In general, the described Mst protein or a nucleotide sequence coding for the Mst protein is brought into contact with a test compound and the influence of the test compound on the Mst protein is measured or detected.

According to the present invention the term “Mst protein modulator” means a modulating molecule (“modulator”) of the biological activity of the Mst protein, in particular an inhibitory or activating molecule (“inhibitor” or “activator”), especially an inhibitor of the Mst protein identifiable according to the assay of the present invention. An inhibitor generally is a compound that, e.g. bind to, partially or totally block activity, decrease, prevent, delay activation, inactivate, desensitize, or down-regulate the activity or expression of at least one of the Mst protein as preferably described above in detail, in particular of the human Mst 1 protein. An activator generally is a compound that, e.g. increase, open, activate, facilitate, enhance activation, sensitize, agonize, or up-regulate the activity or expression of at least one of the Mst proteins as preferably described above in detail, in particular of the human Mst 1 protein. Such modulators include naturally occurring or synthetic ligands, antagonists, agonists, peptides, cyclic peptides, nucleic acids, antibodies, antisense molecules, ribozymes, small organic molecules and the like.

In another preferred embodiment of the present invention the thromboembolic disorder mentioned above is caused by activation and/or aggregation of platelets. According to the present invention the thromboembolic disorder is selected from myocardial infarction, unstable angina, acute coronary syndromes, coronary artery disease, restenosis, stroke, transient ischemic attacks, pulmonary embolism, left ventricular dysfunction, secondary prevention of clinical vascular complications in patients with cardiovascular and/or cerebrovascular diseases, atherosclerosis and/or co-medication to vascular interventions, in particular stroke, myocardial infarction, atherosclerosis and/or restenosis.

The present invention is also directed to a method of screening for a modulator of the Mst protein or the nucleotide sequence coding for the Mst protein, wherein the method comprises the steps of:
(a) contacting a Mst protein or the nucleotide sequence coding for the Mst protein in an assay selected from a thrombosis-related assay, a kinase assay and/or a reporter based cell system assay with a test compound, and
(b) measuring or detecting the influence of the test compound on the activity of a Mst protein.

In one preferred embodiment the thrombosis-related assay is a thrombocyte aggregation assay wherein the thrombocyte aggregation is induced by an inducer such as ADP, Thrombin, TRAP, collagen, convulxin, calciumionophor and/or ristocetin.

In particular, it is preferred to use an in vitro kinase assay, e.g. the ATP consumption assay as described above and in the Examples. Preferred assay conditions are in the presence of MnCl₂, in particular in a concentration of 2 mM MnCl₂. An optimal buffer is e.g. 40 mM Hepes at pH 7.5.

Another preferred assay is the homogeneous Fluorescence polarization (FP)-based assay. FP-based assays are assays, which use polarized light to excite fluorescent substrate peptides in solution. These fluorescent peptides are free in solution and tumble, causing the emitted light to become depolarized. When the substrate peptide binds to a larger molecule, however, such as (P)-Tyr, its tumbling rates are greatly decreased, and the emitted light remains highly polarized. For a kinase assay there are generally two options:
(a) a fluorescent phosphopeptide tracer is bound to a (P)-specific antibody. Phosphorylated products will compete the fluorescent phosphopeptide from the antibody resulting in a change of the polarization from high to low.
(b) a phosphorylated substrate peptide binds to the phosphospecific antibody resulting in a change of polarization from low to high.

Also preferred is the off-chip incubation mobility shift assay which uses a microfluidic chip to measure the conversion of a fluorescent peptide substrate to a phosphorylated product. In this assay the reaction mixture from a microtiter plate well is slipped through a capillary onto the chip, where the peptide substrate and the phosphorylated product are separated by electrophoresis and detected via laser-induced fluorescence. Hereby the signature of the fluorescent signal reveals the extent of the reaction. Such an assay is e.g. commercially available from Caliper LifeSciences, Hopkinton, Mass., USA under the name LabChip® Assay for Mst2.

Another assay preferred for secondary screening is the reporter based cell system assay where, following overexpression of the naturally occurring or a mutant Mst1 gene, the effects of perturbations on activate or inactive endogenous pathways can be detected by measuring effects on gene expression of downstream reporter genes. Such a reporter gene could be—for example—luciferase under the control of promoter elements induced by transcription factors such as NFκB, AP1, or p53. Reporter genes and Mst1 can be co-expressed in cell lines such as Hela or HEK293 cells. Preferably, the cells are seeded onto a well of a multi-well test plate. Examples of similar cell-based assays are described in Hill, S. J. et al. (2001) Curr. Opin. Pharmacol., 1, 526-532 and Hexdall, L. et al. (2001) Biotechniques, 1134-8, 1140. An overview of the most frequently used reporter genes and detection methods can be found in Bronstein, I. et al. (1994) Anal. Biochem., 219, 169-181.

In addition to the above described kinase assays, the following heterogeneous and homogeneous assays can also be used for the determination of the kinase activity of the Mst protein or Mst mutants:

The heterogeneous assays encompass e.g. an ELISA (enzyme linked immuno sorbent assay), a DELFIA (dissociation enhanced lanthanide fluoro immuno assay), an SPA (scintillation proximity assay) and a flashplate assay.

ELISA (enzyme linked immuno sorbent assay)-based assays are offered by various companies. The assays employ random peptides that can be phosphorylated by a kinase, such as Mst. Kinase-containing samples are usually diluted into a reaction buffer containing e.g. ATP and requisite cations and then added to plate wells. Reactions are stopped by simply removing the mixtures. Thereafter, the plates are washed. The reaction is initiated e.g. by the addition of a biotinylated substrate to the kinase. After the reaction, a specific antibody is added. The samples are usually transferred to pre-blocked protein-G plates and after washing e.g streptavidin-HRP is added. Thereafter, unbound streptavidin-HRP (horseradish peroxidase) is removed, the peroxidase colour reaction is initiated by addition of the peroxidase substrate and the optical density is measured in a suitable densitometer.

DELFIA (dissociation enhanced lanthanide fluoro immuno assay)-based assays are solid phase assay. The antibody is usually labelled with Europium or another lanthanide and the Europium fluorescence is detected after having washed away un-bound Europium-labelled antibodies.

SPA (scintillation proximity assay) and the flashplate assay usually exploit biotin/avidin interactions for capturing radiolabeled substrates. Generally the reaction mixture includes the kinase, a biotinylated peptide substrate and γ-[P³³]ATP. After the reaction, the biotinylated peptides are captured by streptavidin. In the SPA detection, streptavidin is bound on scintillant containing beads whereas in the flashplate detection, streptavidin is bound to the interior of the well of scintillant containing microplates. Once immobilized, the radiolabeled substrate is close enough to the scintillant to stimulate the emission of light.

The homogeneous assays encompass e.g. a TR-FRET (time-resolved fluorescence resonance energy transfer) assay, a FP (fluorescence polarization) assay, as already described above, an ALPHA (amplified luminescent proximity homogenous assay), an EFC (enzyme fragment complementation) assay.

TR-FRET (time-resolved fluorescence resonance energy transfer)-based assays are assays, which usually exploit the fluorescence resonance energy transfer between Europium and APC, a modified allophycocyanin or other dyes with overlapping spectra such as Cy3/Cy5 or Cy5/Cy7 (Schobel, U. et al. (1999) Bioconjugate Chem. 10, 1107-1114). After excitation e.g. of Europium with light at 337 nm, the molecule fluoresces at 620 nm. But if this fluorophore is close enough to APC, the Europium will transfer its excitation energy to APC, which fluoresces at 665 nm. The kinase substrate is usually a biotin-labeled substrate. After the kinase reaction, Europium-labeled-(P)-specific antibodies are added along with streptavidin-APC. The phosphorylated peptides bring the Europium-labeled antibody and the streptavidin-APC into close contact. The close proximity of the APC to the Europium fluorophore will cause a quenching of the Europium fluorescence at benefit of the APC fluorescence (FRET).

ALPHA (amplified luminescent proximity homogenous)-based assays, are assays, which rely on the transfer of singlet oxygen between donor and acceptor beads brought into proximity by a phosphorylated peptide. Upon excitation at 680 nm, photosensitisers in donor beads convert ambient oxygen to singlet-state oxygen, which diffuses up to a distance of 200 nm. Chemiluminescent groups in the acceptor beads transfer energy to fluorescent acceptors within the bead, which then emits light at approximately 600 nm.

EFC (enzyme fragment complementation)-based assays or equivalent assays can be used in particular for high-throughput screening of compounds. The EFC assay is based on an engineered β-galactosidase enzyme that consists of two fragments—the enzyme acceptor (EA) and the enzyme donor (ED). When the fragments are separated, there is no β-galactosidase activity, but when the fragments are together they associate (complement) to form active enzyme. The EFC assay utilizes an ED-analyte conjugate in which the analyte may be recognized by a specific binding protein, such as an antibody or receptor. In the absence of the specific binding protein, the ED-analyte conjugate is capable of complementing EA to form active β-galactosidase, producing a positive luminescent signal. If the ED-analyte conjugate is bound by a specific binding protein, complementation with EA is prevented, and there is no signal. If free analyte is provided (in a sample), it will compete with the ED-analyte conjugate for binding to the specific binding protein. Free analyte will release ED-analyte conjugate for complementation with EA, producing a signal dependent upon the amount of free analyte present in the sample.

In a preferred embodiment the above-described assays comprise a further step of selecting a test compound with an activity against a thromboembolic disorder by comparing the changes in the assay in the presence and in the absence of the test compound.

As already described above the Mst protein, in particular the Mst 1 and/or Mst 2 protein, is a human Mst protein.

Generally the test compound is provided in the form of a chemical compound library. For the screening of chemical compound libraries, the use of high-throughput assays are preferred which are known to the skilled person or which are commercially available. According to the present invention the term “chemical compound library” means a plurality of chemical compounds that have been assembled from any of multiple sources, including chemically synthesized molecules and natural products or combinatorial chemical libraries. Advantageously the method of the present invention is carried out on an array and/or in a robotics system e.g. including robotic plating and a robotic liquid transfer system, e.g. using microfluidics, i.e. channeled structured. Preferably, the test compound detected is an inhibitor of platelet activation and/or platelet aggregation, in particular the test compound detected reduces the risk for thrombus formation and/or blood clotting.

Another embodiment of the present invention is directed to a method for producing a medicament for the treatment of a thromboembolic disorder, wherein the method comprises the steps of:
(a) carrying out the method described above,
(b) isolating a detected test compound suitable for the treatment of a thromboembolic disorder, and
(c) formulating the detected test compound with one or more pharmaceutically acceptable carriers or auxiliary substances.

Preferably said thromboembolic disorder is caused by activation and/or aggregation of platelets and in particular selected from a thromboembolic disorder as described above.

For the production of a medicament the pharmaceutically active compound or its pharmaceutically acceptable salt is in a pharmaceutical dosage form in general consisting of a mixture of ingredients such as pharmaceutically acceptable carriers or auxiliary substances combined to provide desirable characteristics together with the pharmaceutically active compound.

The formulation generally comprises at least one suitable pharmaceutically acceptable carrier or auxiliary substance. Examples of such substances are demineralized water, isotonic saline, Ringer's solution, buffers, organic or inorganic acids and bases as well as their salts, sodium chloride, sodium hydrogencarbonate, sodium citrate or dicalcium phosphate, glycols, such a propylene glycol, esters such as ethyl oleate and ethyl laurate, sugars such as glucose, sucrose and lactose, starches such as corn starch and potato starch, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils such as groundnut oil, cottonseed oil, corn oil, soybean oil, caster oil, synthetic fatty acid esters such as ethyl oleate, isopropyl myristate, polymeric adjuvans such as gelatin, dextran, cellulose and its derivatives, albumins, organic solvents, complexing agents such as citrates and urea, stabilizers, such as protease or nuclease inhibitors, preferably aprotinin, ε-aminocaproic acid or pepstatin A, preservatives such as benzyl alcohol, oxidation inhibitors such as sodium sulphite, waxes and stabilizers such as EDTA. Colouring agents, releasing agents, coating agents, sweetening, flavouring and perfuming agents, preservatives and antioxidants can also be present in the composition. The physiological buffer solution preferably has a pH of approx. 6.0-8.0, especially a pH of approx. 6.8-7.8, in particular a pH of approx. 7.4, and/or an osmolarity of approx. 200-400 milliosmol/liter, preferably of approx. 290-310 milliosmol/liter. The pH of the medicament is in general adjusted using a suitable organic or inorganic buffer, such as, for example, preferably using a phosphate buffer, tris buffer (tris(hydroxymethyl)aminomethane), HEPES buffer ([4-(2-hydroxyethyl)piperazino]ethanesulphonic acid) or MOPS buffer (3-morpholino-1-propanesulphonic acid). The choice of the respective buffer in general depends on the desired buffer molarity. Phosphate buffer is suitable, for example, for injection and infusion solutions. Methods for formulating a medicaments as well as suitable pharmaceutically acceptable carrier or auxiliary substance are well known to the one of skill in the art. Pharmaceutically acceptable carriers and auxiliary substances are a. o. chosen according to the prevailing dosage form and identified compound.

The pharmaceutical composition can be manufactured for e.g. oral, nasal, parenteral or topic administration. Parental administration includes subcutaneous, intracutaneous, intramuscular, intravenous or intraperitoneal administration.

The medicament can be formulated as various dosage forms including solid dosage forms for oral administration such as capsules, tablets, pills, powders and granules, liquid dosage forms for oral administration such as pharmaceutically acceptable emulsions, microemulsions, solutions, suspensions, syrups and elixirs, injectable preparations, for example, sterile injectable aqueous or oleaginous suspensions, and dosage forms for topical or transdermal administration such as ointments, pastes, creams, lotions, gels, powders, solutions, sprays, inhalants or patches.

The specific therapeutically effective dose level for any particular patient will depend upon a variety of factors including the activity of the identified compound, the dosage form, the age, body weight and sex of the patient, the duration of the treatment and like factors well known in the medical arts.

The total daily dose of the compounds of this invention administered to a human or other mammal in single or in divided doses can be in amounts, for example, from about 0.01 to about 100 mg/kg body weight or more preferably from about 50 to about 75 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of the present invention per day in single or multiple doses.

The following figures, Tables Sequences and Examples shall explain the present invention without limiting the scope of the invention.

### DESCRIPTION OF THE FIGURES

FIG. 1 shows the gel analysis of fractions enriched for phosphoproteins.
Lane A: Resting platelets;
Lane B: Thrombin-stimulated platelets;
Arrows indicate the positions where Mst homologs have been found by LC-MS/MS identification. In bands A and B, Sok1 was detected, while peptides for Mst1 and Mst2 were identified in the upper and the lower band C (see also Table 1).

FIG. 2 shows an Example for a MS/MS spectrum of the Mst1/2 specific peptide AGNILLNTEGHAK listed in Table 1;

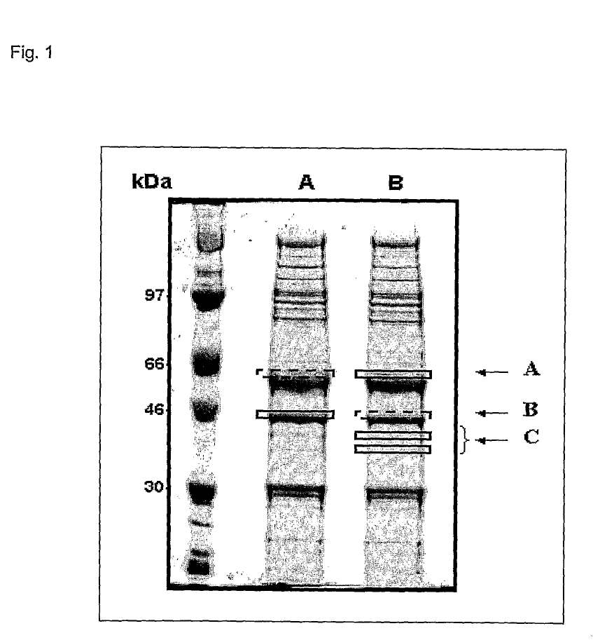

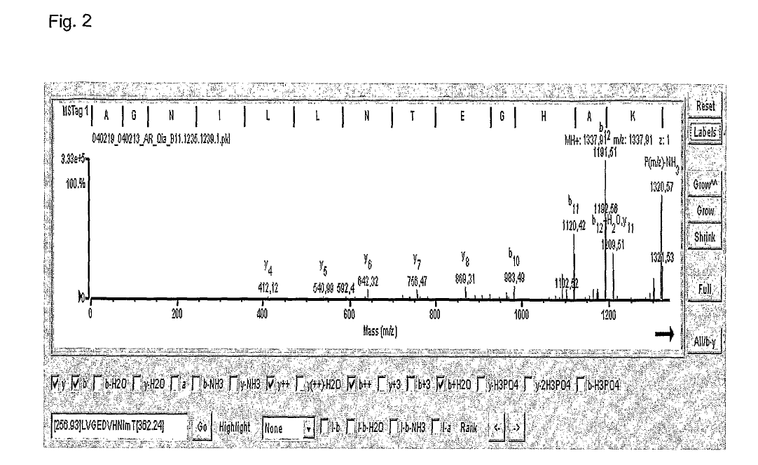

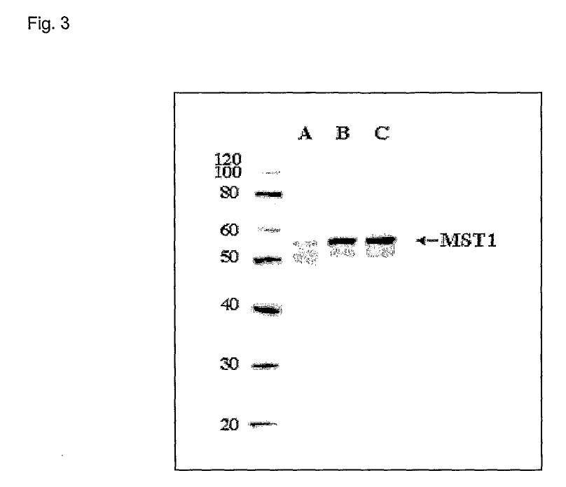

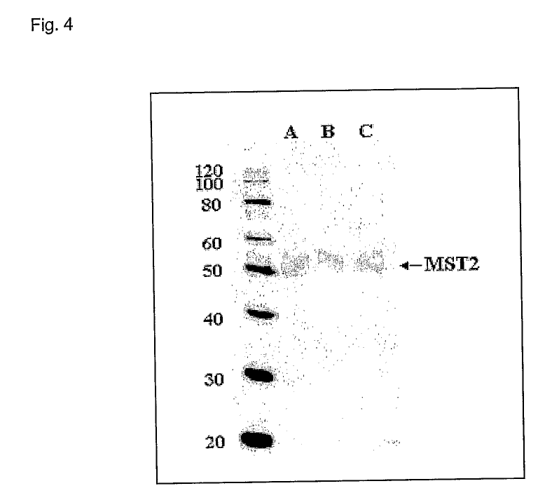

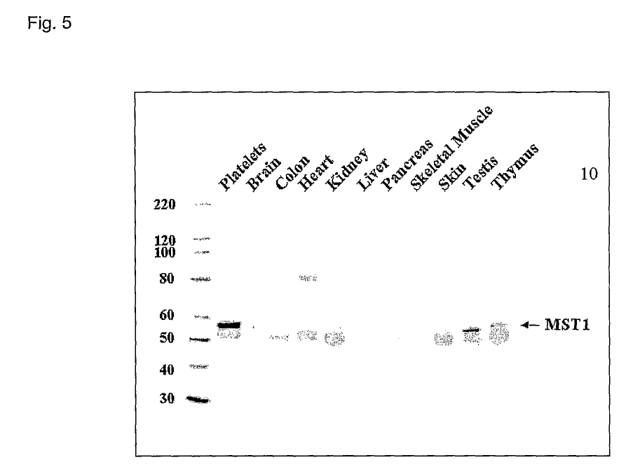

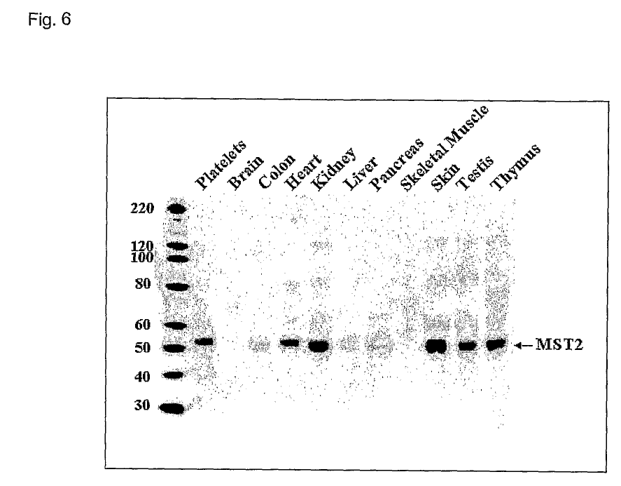

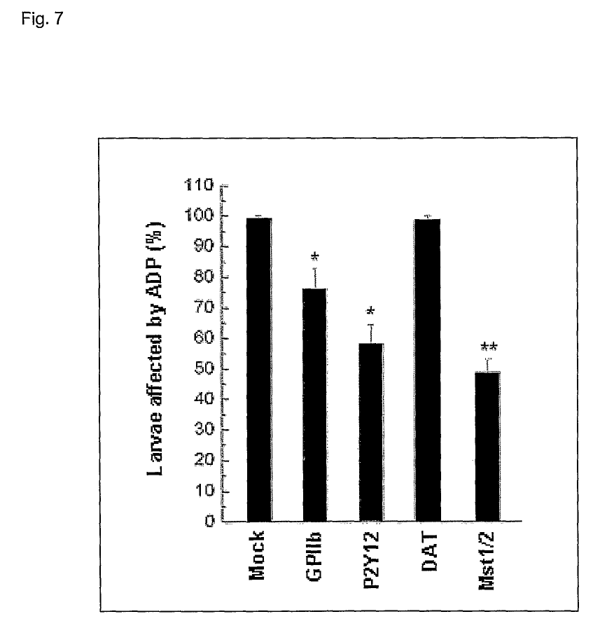

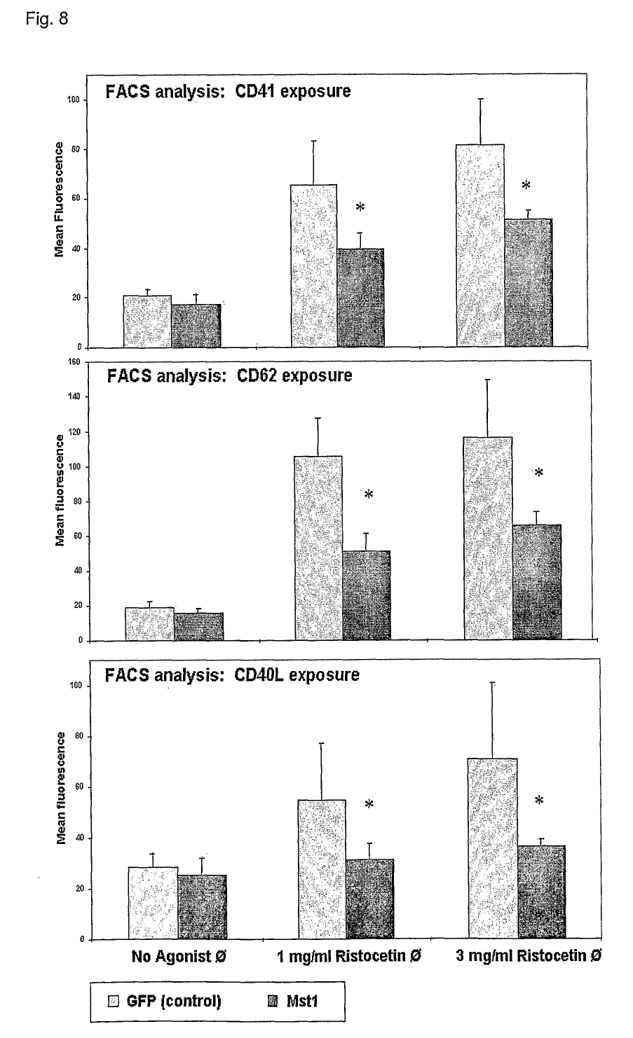

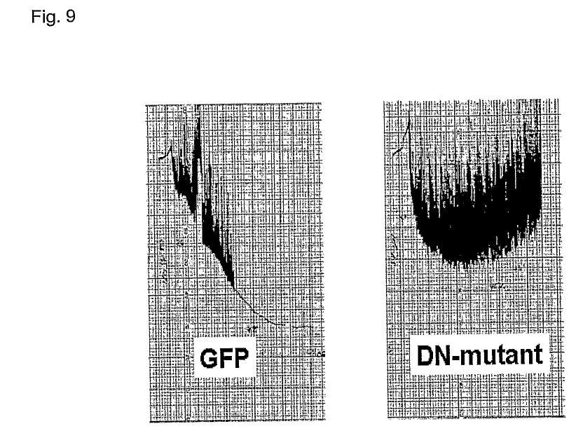

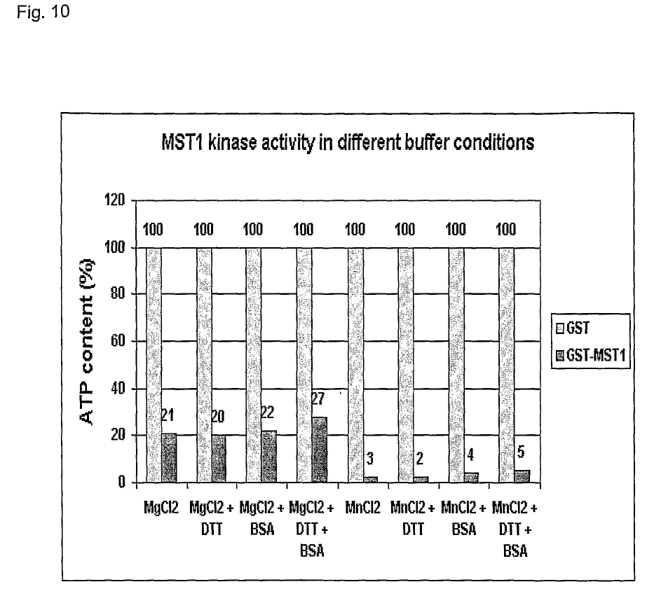

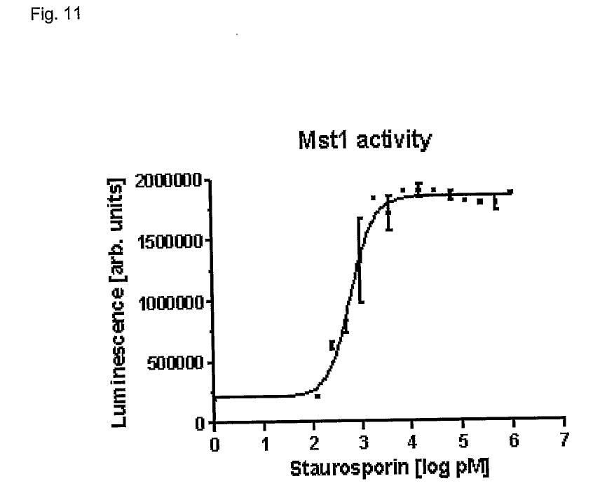

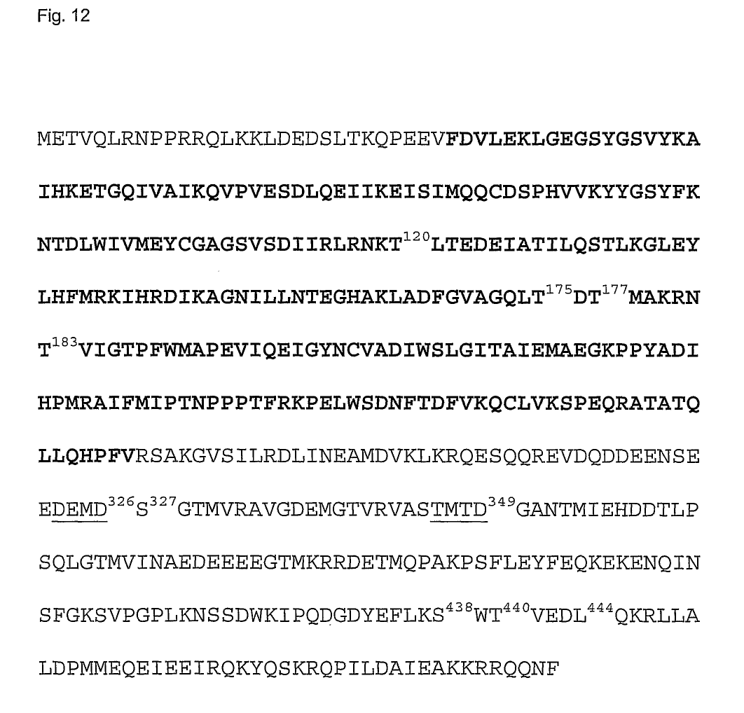

FIG. 3 shows the expression of Mst1 in extracts of testis and human platelets from individual donors.
Lane A: Testis;
Lane B: Resting platelets;
Lane C: Thrombin-activated platelets;
Extracts of testis and human platelets corresponding to 30 μg of total protein were separated by SDS-PAGE and proteins were transferred to nitrocellulose membranes by Western blotting. Mst1 was detected by using a specific antibody.

FIG. 4 shows the expression of Mst2 in extracts of human platelets and testis,
Lane A: Testis;
Lane B: Resting platelets;
Lane C: Thrombin-activated platelets;
Extracts of testis and human platelets corresponding to 30 μg of total protein were separated by SDS-PAGE and proteins were transferred to nitrocellulose membranes by Western blotting. Mst2 was detected by using a specific antibody.

FIG. 5 shows the expression of Mst1 in extracts of various human tissues. 30 μg of total protein from platelets, brain, colon, heart, kidney, liver, pancreas, skeletal muscle, skin, testis, and thymus were separated by SDS-PAGE and proteins were transferred to nitrocellulose membranes by Western blotting. Mst1 was detected by using a specific antibody.

FIG. 6 shows the expression of Mst2 in extracts of various human tissues. 30 μg of total protein from platelets, brain, colon, heart, kidney, liver, pancreas, skeletal muscle, skin, testis, and thymus were separated by SDS-PAGE and proteins were transferred to nitrocellulose membranes by Western blotting. Mst2 was detected by using a specific antibody.

FIG. 7 summarizes the results of an in vivo transgenic zebrafish thrombosis assay and shows that the knockdown of BC048033 (Mst1/Mst2) affects ADP-induced thrombocyte aggregation. BC048033 (Mst1/Mst2) is therefore important for thrombosis in zebrafish.
The assay utilized a transgenic thrombocyte-specific fluorescent zebrafish line, generated by Zygogen using its proprietary Z-Tag™ technology to specifically label thrombocytes with green reef coral fluorescent proteins. Randomized, blinded samples of antisense morpholinos were injected into one cell stage homozygous Z3 embryos. All samples were injected with equi-volume (4 mL) into Z3 fertilized eggs, where the control was 0.2% phenol red, GPIIb was 1.7 ng, P2Y12, an ADP-receptor, was 1.7 ng, DAT (dopamine transporter) was 1.7 ng, and Mst1/2 was 16.6 ng. The embryos were placed in 28° C. incubator. At 6 dpf (days post fertilization), the developed larvae were injected with ADP (approximately 90 pmol) into the heart cavity. Larvae were recorded as either having any or no thrombocyte movement. The data were pooled and averaged. At least 60 larvae in at least 6 independent experiments were analyzed for each condition. Each condition was compared to mock-injected larvae that were treated with ADP. Statistical significance (*=p<0.01, **=p<0.001) was observed for all samples with respect to the control except for DAT.

FIG. 8 shows examples for FACS analyses of ristocetin-stimulated CD-platelets: FACS measurement of recombinant, retrovirally infected CD-platelets expressing either the dominant negative Mst1 mutant Mst1^{K59R} fused to GFP (“Mst1”) or GFP only (“GFP (control)”). Shown are relative increases (%) of mean fluorescence values over basal values. The figure shows the agonist-dependent surface expression of CD41, CD62P (P-selectin), and CD40L respectively. The figure indicates the results from 12 independent experiments done with CD-platelets from 4 independent isolations of megakaryocytes with standard deviations. *p< 0.05.

FIG. 9 shows the original recording of aggregations as induced by thrombin. Representative aggregation tracings of recombinant, retrovirally infected CD-platelets expressing either the dominant negative Mst1 mutant Mst1^{K59R} fused to GFP (“DN-mutant”) or the GFP control only (“GFP”) in response to 0.5 U/mL thrombin.

FIG. 10 shows an GST-Mst1 kinase assay in different buffer conditions. Compared to a GST-control, the highest kinase activity of GST-Mst1, measured as ATP consumption, was obtained in presence of 2 mM MnCl₂.

FIG. 11 shows the sigmoidal dose-response curve of staurosporine on MST1 kinase activity. The effect of each dose was tested on triplicate samples. Staurosporine could inhibit completely in an in vitro assay the ATP consumption due to MST1 kinase activity. The EC50 derived from the best-fit values was 608.1 pM.

FIG. 12 shows SEQ ID NO: 1, the amino acid sequences of human Mst 1, wherein in bold face the Mst 1 kinase domain is shown. The underlined amino acids show the caspase cleavage sites and the numbers indicate examples of point mutations which do not abolish the Mst1 kinase activity.

### DESCRIPTION OF THE SEQUENCES

SEQ ID NO: 1 shows the amino acid sequence of human Mst 1 (Swissprot entry Q13043);
SEQ ID NO: 2 shows a nucleotide sequence coding for human Mst 1 (Genbank entry NM_{—}006282);
SEQ ID NO: 3 shows the amino acid sequence of human Mst 2 (Swissprot entry Q13188);
SEQ ID NO: 4 shows a nucleotide sequence coding for human Mst 2 (Genbank entry NM_{—}006281);
SEQ ID NO: 5 shows the human dominant-negative Mst1: Mst1^{K59R}
SEQ ID NO: 6 shows the Mst1 & Mst2 homolog in Zebrafish (NCBI entry AAH48033; nucleotide sequence corresponding to BC048033)
SEQ ID NO: 7-10 show SOK-1 and Mst-specific peptides as listed in Tab. 1

TABLE 1
Mst-specific peptides which were identified
in a phosphoproteomics approach

| | | Thrombin- | |
|---|---|---|---|
| | Resting | activated | |
| Peptide | platelets | platelets | Protein |
| LADFGVAGQLTDTKQIK | + | + | SOK-1 |
| TLIEDEIATILK | − | + | Mst2 |
| AGNILLNTEGHAK | − | + | Mst1 and/or Mst2 |
| ATATQLLQHPFVR | − | + | Mst1 |

TABLE 2
Expression pattern of human Mst1 mRNA and
Mst2 mRNA in various human tissues.

Expression level

| Tissue Type | Mst1 | Mst2 |
|---|---|---|
| Adrenal Gland | low | low |
| Bone marrow | medium | low |
| Brain | high | low |
| Colon | low | low |
| Fetal Brain | high | low |
| Fetal Liver | medium | low |
| Heart | low | low |
| Kidney | low | low |
| Liver | low | low |
| Lung | low | low |
| Mammary Gland | low | low |
| Pancreas | low | low |
| Placenta | low | low |
| Prostate | low | low |
| Salivary Gland | low | low |
| Skeletal Muscle | low | low |
| Small Intestine | low | low |
| Spinal Cord | low | low |
| Spleen | low | low |
| Stomach | low | low |
| Testis | high | high |
| Thymus | medium | low |
| Thyroid | low | low |
| Trachea | low | low |
| Uterus | low | low |

Mst1 and Mst2 mRNAs were detected by quantitative RT-PCR (Taqman) using specific primers.

### EXAMPLES

### Material & Methods

### Reagents:

Tissue homogenates, provided in a buffer including HEPES (pH7.9), MgCl₂, KCl, EDTA, Sucrose, Glycerol, Sodium deoxycholate, NP-40, and a cocktail of protease inhibitors were purchased from “BioCat GmbH, Heidelberg”. Antibodies against Mst1 and Mst2 were obtained from “Cell Signaling Technology, Inc., Beverly, USA”, “Santa Cruz Biotechnology, Inc., Santa Cruz, USA”, and “Upstate (distributed by Biomol GmbH, Hamburg)”.

### Isolation and Activation of Platelets for Western Blotting Analysis:

Freshly drawn blood from healthy donors was collected in acid-citrate-dextrose formula A (ACD-A) solution (Fresenius Hemocare, Redmond, USA). The blood was centrifuged at room temperature for 20 minutes at 150 g and platelet-rich plasma (PRP) was recovered. 1 volume of ACD-A solution was added to 9 volumes of PRP. Following an additional centrifugation at 120 g for 15 minutes, the PRP was treated with 0.5 ug/ml PgE1 (SIGMA-ALDRICH, Taufkirchen) and platelets were pelleted by centrifuging 15 minutes at 360 g. After resuspension in Tyrode's solution (137 mM NaCl, 2.7 mM KCl, 12 mM NaHCO₃, 0.36 mM NaH₂PO₄, 1 mM MgCl₂, 10 mM Hepes, 5.5 mM Glucose, 0.1% BSA, pH7.4), platelets were either left untreated or treated with 1 U/ml human Thrombin (SIGMA-ALDRICH, Taufkirchen) for 1 to 5 min at room temperature, and pelleted at 360 g for 15 minutes.

### Western Blot Analysis:

Platelet total SDS lysates for protein expression analysis by Western Blotting were prepared as following: thrombocyte pellets, obtained as described above, were resuspended in ice-cold lysis buffer containing 20 mM Hepes, 100 mM NaCl, 1% SDS, 1 mM Na₃VO₄, 10 mM NaF pH7.4, 5 mM EDTA and supplemented with Complete protease inhibitor cocktail (Roche Diagnostics GmbH, Mannheim). Lysates were rolled over top 20 min at 4° C. before being centrifuged 20 minutes at 4° C. at 13000 RPM in a tabletop microcentrifuge. Protein concentration of the supernatant was determined and 30 ug proteins were resolved on either a 4-12% NuPAGE® (in the case of tissue distribution analysis) or a 10% NuPAGE® gel (Invitrogen GmbH, Karlsruhe) and transferred onto a nitrocellulose membrane (Amersham Biosciences Europe GmbH, Freiburg). For detection of Mst1 a rabbit polyclonal antibody anti-Mst1 (Cell Signaling Technology, Inc., Beverly, USA) or a rabbit polyclonal antibody anti-Mst1/Krs-2 (Upstate (distributed by Biomol GmbH, Hamburg) were used. For detection of Mst2 the goat polyclonal antibody Krs-1 (N-19) (Santa Cruz Biotechnology, Inc., Santa Cruz, USA) was used. Briefly, blots were blocked in TBS-T (20 mM Tris-Cl pH7.6, 137 mM NaCl, 0.1% Tween 20 (SIGMA-ALDRICH, Taufkirchen) containing 5% fat-free dried milk rocking o/n at 4° C., and probed with one of the rabbit polyclonal antibodies anti-Mst1 or with the goat polyclonal antibody Krs-1 (N-19) in TBS-T containing 5% fat-free dried milk. After incubation with peroxidase-conjugated secondary antibodies (Jackson ImmunoResearch Europe Ltd., Cambridgeshire, UK and SIGMA-ALDRICH, Taufkirchen), detection was performed by incubating the membranes with the Lumi-Light Western Blotting Substrate according to the manufacturer's instructions (Roche Diagnostics GmbH, Mannheim).

### Phosphoprotein Enrichment:

For selective enrichment Qiagen's PhosphoProtein Purification kit was used as follows: platelet sediments (resting/Thrombin-activated) were resuspended in 3 ml Qiagen Lysis Buffer (QLB) and centrifuged (30 min, 13000×g). Protein concentration of the supernatant was determined using a BCA-Kit (Perbio Science Deutschland GmbH, Bonn) and the protein concentration was adjusted to 2.5 mg/25 ml in QLB.

After equilibrating the Phospho-Protein-Purification-Column with 4 ml of QLB, the platelet solution was applied to the top of the column in 2 portions. The flow-through fraction was collected, and further 6 ml of QLB were applied to wash the column. After that, 500 ul Qiagen Elution Buffer were applied, and the eluate fractions were collected. The elution was repeated 4 times, resulting in 5 eluate fractions. Protein determination was done for all collected fractions, indicating the highest protein amount in fraction 3.

After TCA-precipitation, 30 ug of each respecting fraction 3 (resting/Thrombin-activated) was loaded onto a 1D-Gel (Invitrogen GmbH, Karlsruhe, Nupage® 10%). Colloidal coomassie blue stain was performed according to Roth (Carl Roth GmbH + Co., Karlsruhe).

### Protein Identification

In-gel protein digestion: Coomassie Blue-stained protein bands were excised and cut into small pieces of about 1-2 mm³ in diameter. Destaining was done by washing three times for 15-30 min in 100 μl washing solution (50% acetonitrile/25 mM NH4 HCO₃, pH 8.0). The gel pieces were dried by adding 50 μl acetonitrile; excess solution is removed after about 10 min. Gel pieces are then rehydrated in 15 μl of trypsin solution (5 μg/ml) (rec., proteomics grade, Roche Diagnostics GmbH, Mannheim m) and incubated at 37° C. over night in a convection oven. Peptides were then extracted by incubation with 30 μl 50% acetonitrile/5% TFA (three times). Peptide extracts were pooled, lyophilized in a vacuum centrifuge and reconstituted in 13 μl 0.1% TFA.

Nano-LC-MS/MS Analysis: Analyses of the peptide samples were performed on a nano-ESI-LC-MS/MS system consisting of a Ultimate HPLC system (LC Packings, Amsterdam) coupled to a LCQ Deka XP mass spectrometer (Thermo Finnigan, San Jose). A Famos autoloader (LC Packings, Amsterdam) was used to inject a sample volume of 13 μl. The sample was desalted on a C18 precolumn (PepMap, i.d. 300 μm, 5 mm length) using a Switchos module (LC Packings, Amsterdam); loading and washing of the sample with 2% acetonitrile/0.1% trifluoracetic acid was performed at a flow rate of 30 μL/min for 10 min. A C18 nanocolumn (PepMap, i.d 75 μm, 150 mm length, LC Packings, Amsterdam) was used to separate the peptides at a flow rate of 200 nL/min. The mobile phase consisted of 2% acetonitrile/0.1% formic acid (solvent A) and 98% acetonitrile/0.1% formic acid (solvent B). A linear gradient from 5% B to 35% B in 45 min, followed by a linear increase to 100% B in 10 min, achieved peptide elution.

The capillary tubing of the nano-LC was connected to the electrospray needle with a MicroTee (Upchurch Scientific, Inc., Oak Harbor, USA) where a voltage of 1.2 kV was applied. Nano-electrospray needles were laboratory-pulled (Sutter Instruments Co., Novato, USA, Model P-2000) from fused silica capillaries (i.d. 25 μm, o.d. 280 μm, Grom Chromatography GmbH, Rottenburg-Hailfingen) resulting in a needle orifice of approximately 3 μm in diameter.

Mass spectrometry analyses were controlled by the XCalibur software (Thermo Finnigan, San Jose) using data dependent acquisition in the positive ion mode. The transfer capillary temperature was constantly held at 180° C., the capillary voltage and the tube lens offset on 46 V and 55 V, respectively. Peptides eluted from the column were detected in a first scan event in MS mode (m/z 500-2000, 3 microscans, maximum injection time 50 ms), followed by three consecutive data dependent MS/MS scan events (isolation width 3 Da, 4 microscans, maximum injection time 400 ms, activation time 30 ms) for the three most abundant ions (above 5×10⁵ counts) using a relative collision energy of 35% (corresponding to the XCalibur software settings). The dynamic exclusion parameters were set as follows: “repeat count” 2, “repeat duration” 0.5 min, “exclusion list size” 25, “exclusion mass width”+/−1.5 Da, “exclusion duration” 1.50 min, no rejections.

Mass spectrometry: Mass data were processed with SpectrumMill. The following parameters were used to convert raw data into .pkl-files: no “cystein modification”, “minimal sequence tag”> 1, “scan range” 1-9999 (all), “[M+H]⁺” 500-4000 Da, “parent charge assignment” find force 1 through 4/find max (z) 7/min MS S/N 25, “merge scans with the same parent” m/z+/−1 scan (no merging of spectra). Protein identifications were obtained by searching the SwissProt database while the taxonomy was restricted to mammals. Searches were done with matching tolerances of +/−1.5 Da and +/−0.7 Da for the parent and the fragment masses, respectively. A maximum number of 2 missed tryptic cleavages was allowed. A peptide sequence tag was regarded as reliable when the identification met the following parameters set in the validation filter: “protein score” >8, “peptide score” >8, “% SPI” >70; in addition all spectra were manually examined.

### Zebrafish Analyses:

For target validation in the Z-Tag thrombosis assay, antisense morpholinos designed to recognize the first 25 nucleotides beginning at the translational start site have been used. Morpholinos were ordered from Gene Tools, Inc. Philomath, USA and tested in the Z-Tag thrombosis assay. The effective concentration of the morpholino was evaluated by injecting several concentrations, ranging from 0.83 ng-33.2 ng, of each morpholino construct into the one cell stage of Z3 embryos. The 6 dpf larvae that did not exhibit any adverse changes in development with respect to the used concentration were tested for their response to ADP. Injected embryos were placed at 28° C. until tested in the thrombosis assay. Approximately 90 pmol of ADP was injected into the heart cavity of morpholino-injected larvae. The ADP-injected larvae were observed 5 min thereafter under fluorescent stereomicroscopy, and the presence or absence of thrombocyte movement was assessed and recorded.

### Generation of CD-Platelets:

Transgenic mouse platelets expressing the dominant-negative mutant of Mst1 were generated according to Ungerer, M. et al. (2004) Circ. Res. 95, e36-e44.

In brief, murine bone marrow cells were harvested by flushing the femurs and tibiae of mice. Megakaryocyte precursor cells from freshly isolated bone marrow were cultured under conditions allowing a large majority of the cells to differentiate into megakaryocytes. The cDNA for the dominant negative mutant of Mst1, Mst1^{K59R} was cloned into the plasmid pLEGFP-C1 obtained from Clontech, Heidelberg, Germany. Human GP2-293, a pantropic retroviral packaging cell line, was grown in Dulbecco's modified Eagle's medium (DMEM) with Glutamax (Invitrogen GmbH, Karlsruhe) supplemented with 10% fetal calf serum (PAA, Cölbe, Germany), 1% sodium pyruvate, 100 mmol/L (Biochrom AG, Berlin), 1% pen/strep (Biochrom AG, Berlin). NIH 3T3 cells were maintained in DMEM/Glutamax supplemented with 5% fetal calf serum. 24 hours before transfection, cells were split 1:2 into 150 mm dishes. Transfection was performed by calcium phosphate coprecipitation as available by Clontech with chloroquine (50 mmol/L, SIGMA-ALDRICH, Taufkirchen). 30 hours after transfection, virus was collected every 24 hours for at least 3 days.

After harvesting the virus and determination of the viral titer, megakaryocyte precursors were cultured in 48-well plates in IMDM only with stem cell factor. Two days after isolation, cells were infected with a MOI of 2-5 cfu/cell in the presence of polybrene (8 μg/mL) and DEAE dextrane (1 mg/mL). For coculture experiments, 6×10⁶ transfected GP2-293 producer cells were incubated with 1.5×10⁶ isolated megakaryocytes (2 days after their isolation) in the presence of 10 mL IMDM, 10 mL DMEM, Polybrene and DEAE dextrane. This coculture was incubated for 24 h at 37° C. and the medium with the megakaryocytes was transferred into a 6-well plate for culturing over 4-6 weeks. 36 hours after infection or after the initiation of coculture, the cells received their complete medium (Ungerer, M. et al. (2004) supra) and geneticin (380 μg/mL) to select the infected from the non-infected cells.

Culture-derived platelets were harvested by centrifugation and washed. Depending on the respective protocol, they were activated for 10 min with collagen, thrombin or ristocetin. The antibodies for mouse CD41, CD-40L, CD61 and CD62P were from Pharmingen, Leiden, The Netherlands. Expression of these antigens was evaluated by FACS analysis, as described. For aggregation experiments, a Chrono-Log 500 VS aggregometer was filled with 300 μL platelet-rich plasma or a mix of 10⁷ CD-platelets/mL in modified Tyrode's buffer containing fibrinogen (480 μg/mL) and CaCl₂ (2.5 mmol/L). After adding the respective agonist, light transmission was recorded continuously for the following 20 minutes. Curves induced by thrombin were detected in the presence of 2 mmol/L plasmin inhibitory peptide (GPRP).

### Expression of GST-Mst Proteins in Bacteria and Mst Kinase Assay:

The full-length sequences of the human Mst1 and Mst2 were amplified by RT-PCR starting from RNA of HeLa cells. According to the Invitrogen Gateway® Technology manual, generated PCR products were employed in a BP recombination reaction to obtain entry clones. The sequences were subsequently transferred into pDEST15 for bacterial expression. pDEST15-MST vectors were transformed into the E. Coli BL21-AI strain and grown on Ampicillin for selection of transformants carrying the DNA of interest. Single colonies were inoculated in LB medium containing 100 μg/ml Ampicillin until they reached the appropriate cell density. They were then induced for 2.5 h at 37° C. with 0.2% Arabinose (SIGMA-ALDRICH, Taufkirchen). At the end of the induction, bacteria were washed in ice-cold PBS and resuspended in lysis buffer (137 mM NaCl, 2.7 mM KCl, 10 mM Na₂HPO₄, 1.4 mM KH₂PO₄, 5 mM NaF, 3 mM EDTA, pH7.5 supplemented with 0.25% Triton X-100, 0.25% CHAPS, 1 mM Na₃VO₄ and protease inhibitors). After the bacteria were successfully lysed through a French press, the lysates were cleared by centrifuging 30 minutes at 20000 g at 4° C.

GST recombinant proteins were purified from the cleared lysates using the MagneGST Protein Purification System (Promega GmbH, Mannheim) according to the manufacturers protocol.

To determine optimal conditions for in vitro kinase assay (ATP consumption assay), 1 μg GST (Glutathione-S-Transferase)-Mst proteins were incubated with 2 μg of MBP (Myelin Basic Protein) and 2 μM ATP in kinase buffer containing 40 mM Hepes pH7.5 supplemented with either 10 mM MgCl₂ or 2 mM MnCl₂. In addition, we evaluated the effect on kinase activity of 1 mM DTT and/or 0.02% BSA. The reactions were performed for 30 minutes at 32° C. and terminated by addition of 1 μM Staurosporine in ATP Monitoring Reagent (Cambrex Bio Science Nottingham Ltd, Nottingham, UK) for bioluminescent measurement of ATP consumption compared to a GST control.

To assess the effect of the unspecific kinase inhibitor staurosporine (SIGMA-ALDRICH, Taufkirchen) on Mst1 kinase activity, 1 μg GST-Mst1 protein was pre-incubated for 30 minutes at 32° C. with serial dilutions of staurosporine. Afterwards the kinase assay was performed as described above. Each determination was carried out in triplicate. The success of the treatment was determined based on the decrease in ATP consumption compared to the untreated samples.

### Results

### Initial Identification of Mst2 in Platelets

Mst2 was identified by applying the so-called PST technology to enriched platelet membranes combined with an experimental bioinformatics assignment procedure (Kuhn, K. (2003) J. Proteome Res 2, 598-609), Mst2 was identified using this LC-MS based analysis. Mst2 was identified by 6 PST's with high confidence.

### Identification of Mst Kinases by a Phosphoproteomics Approach

In order to identify signalling related proteins, phosphorylated proteins were enriched from either resting or thrombin-stimulated platelets using IMAC affinity media from Qiagen. Fractions containing the majority of eluted protein were further separated on 1D gels. Whole lanes were then cut into stripes and containing proteins were identified by in-gel trypsin digestion followed by LC-MS/MS analysis. Mst kinases were found at several locations in the gel as illustrated in FIG. 1 and Table 1. In addition to Mst1 and Mst2 also SOK1 as another homolog of Mst1 and 2 was detected (FIG. 1 and Table 1). This is the first time that SOK1 was identified in platelets. SOK1 was identified in two bands migrating at different MW, probably due to proteolytic degradation of SOK1.

The MS data give some first hints about functional informations, as the peptides for Mst1 and Mst2 were found only in Thrombin-activated platelet extracts, which might indicate a stimulus-induced specific phosphorylation of Mst1 and/or Mst2. In these experiments, mainly a protease-cleaved form of Mst1 and Mst2 with an apparent molecular weight of ˜35 kDa could be identified.

### Expression Pattern for Mst1 and Mst2 in Human Tissues

The tissue distribution of Mst1 and Mst2 in different human tissues was first investigated by Taqman analyses.

According to the Taqman analyses, Mst1 mRNA is expressed at high levels in human brain, fetal brain and testis (Table 2). Medium expression levels were found in bone marrow, fetal liver, and thymus. In all other tissues Mst1 mRNA was below the detection limit and therefore not expressed or expressed only at low level. Thus, Mst1 is expressed only in few tissues. In contrast to Mst1, Mst2 mRNA could be detected at high expression levels only in testis. As observed for Mst1, in all other tissues Mst2 mRNA was below the detection limit and thus not expressed or expressed only at low level (Table 2).

Anucleate platelets contain only very little amounts of mRNA in comparison to nucleate cells. Furthermore, mRNA isolated from platelets often is found to be more degraded in comparison to mRNA obtained from nucleate cells. Therefore, platelet mRNA was not included in our Taqman panel, but rather protein extracts and Western blot analysis were used in order to evaluate the protein expression levels of Mst1 and Mst2 in platelets and other tissues.

### Mst1 and Mst2 are Expressed in Human Platelets

To verify the expression of Mst1 and Mst2 in human platelets, thrombocytes from whole blood of individual donors were purified in several steps (see Material & Methods). Consequently, contamination by other cell types as well as by serum components could be avoided. Total cell extracts of platelet pellets were then obtained through lysis in SDS-containing buffer.

At the amino acid level Mst1 and Mst2 have an identity of 77.6%. To discriminate between them by Western Blotting, antibodies whose specificity had been tested on recombinant proteins have been used.

Since the Taqman analysis had indicated very high expression levels of Mst1 mRNA in testis, extracts of human testis were used as positive control for the analysis of Mst1 protein expression by Western Blotting.

As shown in FIG. 3, on protein level Mst1 is expressed weakly also in testis, thus partly confirming the results of the Taqman analysis. Mst1 could be clearly detected in human platelets at expression levels much higher than in testis. This result has been confirmed in several donors.

In view of the fact that also Mst2 mRNA is highly expressed in testis as shown by the Taqman results (Table 2), extracts of human testis were used as positive control for the analysis of Mst2 protein expression by Western Blotting. As shown in FIG. 4, Mst2 is expressed in testis on protein level, thereby confirming the results of the Taqman analysis. In addition, Mst2 is expressed also in human platelets at levels comparable to those observed in testis, but showing some donor-dependent variability in its amount.

### Tissue Distribution of Mst1 and Mst2

The tissue distribution of Mst1 and Mst2 in various human tissue extracts including platelets was investigated.

On protein level, Mst1 is unambiguously expressed in platelets, testis, and thymus, with highest expression levels constantly found in platelets. In the other tissues examined, Mst1 is not expressed (FIG. 5). In some of the samples, and especially in colon, heart, kidney and skin, an additional band at around 50 kDa could be detected. The unspecific nature of this signal has been verified by probing the same samples with a different Mst1-specific antibody (Upstate (distributed by Biomol GmbH, Hamburg): in agreement with the result shown in FIG. 5, Mst1 expression could be confirmed in platelets, testis and thymus, whereas no signal was detected in other tissues (data not shown). In contrast to the Taqman results (Table 2), Mst1 is not detected in the brain on protein level.

On protein level, Mst2 is strongly expressed in several tissues including testis, thymus, and skin (FIG. 6). Medium to strong expression could be detected in platelets, kidney, and heart. Low levels of expression were observed in colon, pancreas and liver (FIG. 6).

Another important finding of these tissue distribution analyses for Mst2 is that the results found on protein level clearly differ from those found on mRNA level, where expression of Mst2 mRNA could be only detected in testis (Table 2).

### Functional Validation for Mst1 and Mst2 in the Zebrafish

An in vivo transgenic zebrafish thrombosis assay for compound screening and for target identification and validation was developed. In the assay thrombocytes (zebrafish equivalent of platelets) with fluorescent proteins were specifically labelled. A thrombosis-related assay using ADP as platelet agonist has been developed using Z-Tag (Zygogen, LLC) by injecting the relevant agonist into the heart cavity of Z-Tag embryos in the TG(GPIIb:G-RCFP)Z3 zebrafish line with fluorescent platelets. Two known and validated platelet targets, the GPIIb receptor and the P2Y₁₂ receptor, are conserved in zebrafish and their role in ADP-induced thrombocyte aggregation was demonstrated using rapid antisense morpholino technology, thereby validating the zebrafish model system.

The morpholino-based antisense technology was employed (Zygogen, LLC) to characterize Mst1 and Mst2 as potential thrombosis target genes. In contrast to the situation in human, the Zebrafish genome contains only one homolog representing both, Mst1 and Mst2. The gene product BC048033 (76.8% and 88.8% identity to human Mst1 and Mst2, respectively) represented the complete zebrafish orthologue. Another potential homolog gene in zebrafish, BC045867, shows a by far higher degree of similarity to yet another human homolog of Mst1 and Mst2, called Mst3 (Schinkmann, K. et al. (1997) J. Biol. Chem. 272, 28695-28703). BC045867 is conserved by ˜76% to human Mst3, but only by ˜45% and ˜47% to human Mst1 and Mst2, respectively. In contrast, BC048033 is conserved to human Mst3 only by ˜45%. A search in the Sanger Center zebrafish genomic database did not identify any other potential zebrafish genes for Mst1 or Mst2, suggesting that the presence of two homologs (Mst1 and Mst2) in human could represent a gene duplication event. Thus, by using antisense technology to eliminate expression of zebrafish BC048033 the expression of Mst1 and Mst2 were knocked down in parallel when comparing to the situation in human.

For target validation in the Z-Tag thrombosis assay, antisense morpholinos designed to recognize the first 25 nucleotides beginning at the translational start site have been shown to be effective. Therefore the 5′ end for BC048033 was determined. Morpholinos were ordered from Gene Tools, Inc. Philomath, USA and tested in the Z-Tag thrombosis assay. The morpholino study was conducted in 2 phases. The first part was a preliminary phase where the effective concentration of each morpholino was evaluated. This was determined by injecting several concentrations, ranging from 0.83 ng-33.2 ng, of each morpholino construct into the one cell stage of Z3 embryos. For the Mst1/Mst2 morpholino, no adverse side effects on the development of the embryo were observed for the concentrations tested. Knock down of the GPIIb receptor and the P2Y₁₂ receptor were used as positive controls for these experiments.

The results of these experiments which were conducted as a blinded study clearly suggest that Mst1/Mst2 is important for ADP-induced thrombocyte aggregation (FIG. 7). In addition, the GPIIb and P2Y₁₂ morpholinos as positive controls were effective in this blind study (FIG. 7). As a negative control, a morpholino directed towards dopamine transporter (DAT) was used. This morpholino has been found to be effective in a neurodegenerative disease model (Zygogen unpublished data). As predicted, the DAT morpholino did not have any effect on ADP-induced thrombocyte aggregation.

BC048033, the Mst1/Mst2 homolog in zebrafish was the best candidate gene within this study. The frequency of its effect on ADP-induced aggregation was greater than what was observed for any other gene, including the GPIIb and P2Y₁₂ receptors. In addition, knockdown of the gene was not lethal and did not cause any delay in development of zebrafish larvae. This may be an indication that there would be few, if any, side effects for drugs targeting this protein, thereby strongly supporting it's potential as target gene for the development of anti-thrombotic drugs.

### Functional Validation for Mst1 in Culture-Derived Platelets

A system for the generation of culture-derived (CD)-platelets from megakaryocyte precursor cells was employed that can be used for the overexpression of target genes and mutants in transgenic CD-platelets (Lingerer, M. et al. (2004) supra). This system has been used for the validation of Mst1 in mouse megakaryocyte precursor cells derived CD-platelets. A dominant-negative mutant of Mst1, Mst1^{K59R} was overexpressed in culture-derived platelets (CD-platelets) with the aim to investigate any changes in the function of these platelets caused by the resulting interference with the activity of native Mst1.

Retroviraly induced overexpression of Mst1^{K59R} did not alter the morphology of megakaryocytes, neither of shedded CD-platelets, nor the expression of megakaryocyte-specific markers compared to GFP only-expressing cells or uninfected control cells. Also the numbers of resulting CD-platelets were similar to those in the other platelet groups. The generated transgene-expressing CD-platelets were used to investigate the activation-dependent expression of surface receptors, the aggregation profile and dense and alpha granule release.

The surface recruitment of fibrinogen receptors after agonist stimulation was tested by investigating expression of CD41 and CD61 on CD-platelets. As shown in FIG. 8, inhibition of Mst1 by the dominant-negative mutant resulted in a marked suppression of the ristocetin-induced surface recruitment of fibrinogen receptor activation markers CD41. Similar results were observed for the surface recruitment of CD61. CD40L was used as a marker for secretion from platelet stores and granules, and alpha degranulation was tested by studying the surface translocation of P-selectin (CD62P). Surface recruitment of CD40L as well as of P-selectin were significantly reduced in the CD-platelets expressing the dominant-negative Mst1 mutant Mst1^{K59R} in response to ristocetin (FIG. 8).

Furthermore, the thrombin-induced aggregation was inhibited in CD-platelets expressing Mst1^{K59R} (FIG. 9). Similarly, the ristocetin-induced aggregation (0.8 mg/ml ristocetin) was inhibited in CD-platelets expressing Mst1^{K59R}.

Altogether, the results in the mammalian culture-derived platelets confirm the functional relevance already demonstrated in the experiments performed in zebrafish. Furthermore, they underline the role for Mst1 as a new target for the development of drugs that will interfere with platelet activation and aggregation.

### 3 Mst1 and Mst2 In Vitro Kinase Assay

To evaluate conditions suitable for an in vitro kinase assay finalized to modulators identification, the in vitro activity of recombinant GST-Mst proteins expressed in bacteria was tested in different buffers. As shown in FIG. 10 for GST-Mst1, in all samples containing 2 mM MnCl₂ and independently on the addition of 1 mM DTT or 0.02% BSA, we observed the highest reduction in ATP content thus corresponding to the highest level of kinase activity. Similar results have been obtained for GST-Mst2, even if the overall ATP consumption never reached the degree observed for GST-Mst1, suggesting a lower kinase activity of the GST-Mst2 recombinant protein.

After establishing optimal buffer conditions for Mst1 and Mst2 (40 mM Hepes pH7.5, 2 mM MnCl₂), we investigated the effect of the unspecific kinase inhibitor staurosporine on GST-Mst1 kinase activity. The GST-Mst1 kinase assay was performed in presence of different concentrations of inhibitor, ranging from 1 μM to 244 pM. As shown in FIG. 11, staurosporine could completely inhibit MST1 activity, with an IC₅₀ of around 600 pM.

These results show that it was possible to optimise the conditions for Mst1 kinase assay. In addition, such conditions were successfully applied to investigate the effect of the kinase inhibitor staurosporine, thereby supporting the application of the assay for use as an in vitro screening assay for compounds that act as modulators of Mst1 and/or Mst2 activity in accordance with the present invention.

### Reporter Based Pathway Mapping

A reporter based cell system for pathway mapping has been established using different cell lines and stimulations. This assay allows investigating the effect of an over-expressed protein of interest on endogenous pathways. By measuring perturbations in the induction of the downstream reporter gene luciferase, whose expression is driven by pathway-specific promoters, the involvement of a protein of interest in a specific signal transduction pathway can be assessed.

It has been shown that, compared to an uninduced control vector, overexpression of Mst1 in HEK293T cells causes a significant activation of the AP-1, NF-κB and p53 promoters. Thus, it will be possible to use such a system where Mst1 is overexpressed and such an inducible promoter is used for readout to test for the cellular effect and efficiency of modulators of Mst1 activity.

SEQ ID NO: 1

METVQLRNPPRRQLKKLDEDSLTKQPEEVFDVLEKLGEGSYGSVYKAIHK
ETGQIVAIKQVPVESDLQEIIKEISIMQQCDSPHVVKYYGSYFKNTDLWI
VMEYCGAGSVSDIIRLRNKTLTEDEIATILQSTLKGLEYLHFMRKIHRDI
KAGNILLNTEGHAKLADFGVAGQLTDTMAKRNTVIGTPFWMAPEVIQEIG
YNCVADIWSLGITAIEMAEGKPPYADIHPMPAIFMIPTNPPPTFRKPELW
SDNFTDFVKQCLVKSPEQRATATQLLQHPFVRSAKGVSILRDLINEAMDV
KLKRQESQQREVDQDDEENSEEDEMDSGTMVRAVGDEMGTVRVASTMTDG
ANTMIEHDDTLPSQLGTMVINAEDEEEEGTMKRRDETMQPAKPSFLEYFE
QKEKENQINSFGKSVPGPLKNSSDWKIPQDGDYEFLKSWTVEDLQKRLLA
LDPMMEQEIEEIRQKYQSKRQPILDAIEAKKRRQQNF

SEQ ID NO: 2

ATGGAGACGGTACAGCTGAGGAACCCGCCGCGCCGGCAGCTGAAAAAGTT
GGATGAAGATAGTTTAACCAAACAACCAGAAGAAGTATTTGATGTCTTAG
AGAAACTTGGAGAAGGGTCCTATGGCAGCGTATACAAAGCTATTCATAAA
GAGACCGGCCAGATTGTTGCTATTAAGCAAGTTCCTGTGGAATCAGACCT
CCAGGAGATAATCAAAGAAATCTCTATAATGCAGCAATGTGACAGCCCTC
ATGTAGTCAAATATTATGGCAGTTATTTTAAGAACACAGACTTATGGATC
GTTATGGAGTACTGTGGGGCTGGTTCTGTATCTGATATCATTCGATTACG
AAATAAAACGTTAACAGAAGATGAAATAGCTACAATATTACAATCAACTC
TTAAGGGACTTGAATACCTTCATTTTATGAGAAAAATACACCGAGATATC
AAGGCAGGAAATATTTTGCTAAATACAGAAGGACATGCAAAACTTGCAGA
TTTTGGGGTAGCAGGTCAACTTACAGATACCATGGCCAAGCGGAATACAG
TGATAGGAACACCATTTTGGATGGCTCCAGAAGTGATTCAGGAAATTGGA
TACAACTGTGTAGCAGACATCTGGTCCCTGGGAATAACTGCCATAGAAAT
GGCTGAAGGAAAGCCCCCTTATGCTGATATCCATCCAATGAGGGCAATCT
TCATGATTCCTACAAATCCTCCTCCCACATTCCGAAAACCAGAGCTATGG
TCAGATAACTTTACAGATTTTGTGAAACAGTGTCTTGTAAAGAGCCCTGA
GCAGAGGGCCACAGCCACTCAGCTCCTGCAGCACCCATTTGTCAGGAGTG
CCAAAGGAGTGTCAATACTGCGAGACTTAATTAATGAAGCCATGGATGTG
AAACTGAAACGCCAGGAATCCCAGCAGCGGGAAGTGGACCAGGACGATGA
AGAAAACTCAGAAGAGGATGAAATGGATTCTGGCACGATGGTTCGAGCAG
TGGGTGATGAGATGGGCACTGTCCGAGTAGCCAGCACCATGACTGATGGA
GCCAATACTATGATTGAGCACGATGACACGTTGCCATCACAACTGGGCAC
CATGGTGATCAATGCAGAGGATGAGGAAGAGGAAGGAACTATGAAAAGAA
CGGATGAGACCATGCAGCCTGCGAAACCATCCTTTCTTGAATATTTTGAA
CAAAAAGAAAAGGAAAACCAGATCAACAGCTTTGGCAAGAGTGTACCTGG
TCCACTGAAAAATTCTTCAGATTGGAAAATACCACAGGATGGAGACTACG
AGTTTCTTAAGAGTTGGACAGTGGAGGACCTTCAGAAGAGGCTCTTGGCC
CTGGACCCCATGATGGAGCAGGAGATTGAAGAGATCCGGCAGAAGTACCA
GTCCAAGCGGCAGCCCATCCTGGATGCCATAGAGGCTAAGAAGAGACGGC
AACAAAACTTCTGA

SEQ ID NO: 3

MEQPPAPKSKLKKLSEDSLTKQPEEVFDVLEKLGEGSYGSVFKAIHKESG
QVVAIKQVPVESDLQEIIKEISIMQQCDSPYVVKYYGSYFKNTDLWIVME
YCGAGSVSDIIRLRNKTLIEDEIATILKSTLKGLEYLHFMRKIHRDIKAG
NILLNTEGHAKLADFGVAGQLTDTMAKRNTVIGTPFWMAPEVIQEIGYNC
VADIWSLGITSIEMAEGKPPYADIHPMRAIFMIPTNPPPTFRKPELWSDD
FTDFVKKCLVKNPEQRATATQLLQHPFIKNAKPVSILRDLITEANEIKAK
RHEEQQRELEEEEENSDEDELDSHTMVKTSVESVGTMRATsTMSEGAQTM
IEHNSTMLESDLGTMVINSEDEEEEDGTMKRNATSPQVQRPSFMDYFDKQ
DFKNKSHENCNQNMHEPFPMSKNVFPDNWKVPQDGDFDFLKNLSLEELQM
RLKALDPMMEREIEELRQRYTAKRQPILDAMDAKKRRQQNF

SEQ ID NO: 4

ATGGAGCAGCCGCCGGCGCCTAAGAGTAAACTAAAAAAGCTGAGTGAAGA
CAGTTTGACTAAGCAGCCTGAAGAAGTTTTTGATGTATTAGAGAAGCTTG
GAGAAGGGTCTTATGGAAGTGTATTTAAAGCAATACACAAGGAATCCGGT
CAAGTTGTCGCAATTAAACAAGTACCTGTTGAATCAGATCTTCAGGAAAT
AATCAAAGAAATTTCCATAATGCAGCAATGTGACAGCCCATATGTTGTAA
AGTACTATGGCAGTTATTTTAAGAATACAGACCTCTGGATTGTTATGGAG
TACTGTGGCGCTGGCTCTGTCTCAGACATAATTAGATTACGAAACAAGAC
ATTAATAGAAGATGAAATTGCAACCATTCTTAAATCTACATTGAAAGGAC
TAGAATATTTGCACTTTATGAGAAAAATACACAGAGATATAAAAGCTGGA
AATATTCTCCTCAATACAGAAGGACATGCAAAATTGGCAGATTTTGGAGT
GGCTGGTCAGTTAACAGATACAATGGCAAAACGCAATACTGTAATAGGAA
CTCCATTTTGGATGGCTCCTGAGGTGATTCAAGAAATAGQCTATAACTGT
GTGGCCGACATCTGGTCCCTTGGCATTACTTCTATAGAAATGGCTQAAGG
AAAACCTCCTTATGCTGATATACATCCAATGAGGGCTATTTTTATGATTC
CCACAAATCCACCACCAACATTCAGAAAGCCAGAACTTTGGTCCGATGAT
TTCACCGATTTTGTTAAAAAGTGTTTGGTGAAGAATCCTGAGCAGAGAGC
TACTGCAACACAACTTTTACAGCATCCTTTTATCAAGAATGCCAAACCTG
TATCAATATTAAGAGACCTGATCACAGAAGCTATGGAGATCAAAGCTAAA
AGACATGACQAACAGCAACGAGAATTGGAAGAGGAAGAAGAAAATTCGGA
TGAAGATGAGCTGGATTCCCACACCATGGTGAAGACTAGTGTGGGAGAGT
GTGQCACCATGCGGGCCACAAGcACGATGAGTGAAGGGGCCCAGACCATG
ATTGAACATAATAGCACGATGTTGGAATCCGACTTGGGGACcATGGTGAT
AAACAGTGAGGATGAGGAAGAAGAAGATGGAACTATGAAAAGAAATGCAA
CCTCACCACAAGTACAAAGACCATCTTTCATGGACTACTTTQATAAGCAA
GACTTCAAGAATAAGAGTCACGAAAACTGTAATCAGAACATGCATGAACC
CTTCCCTATGTCCAAAAACGTTTTTCCTGATAACTGGAAAGTTCCTCAAG
ATGGAGACTTTGACTTTTTGAAAAATCTAAGTTTAGAAGAACTACAGATG
CGGTTAAAAGCACTGGACCCCATGATGGAACGGGAGATAGAAGAACTTCG
TCAGAGATACACTGCGAAAAGACAGCCCATTCTGGATGCGATGGATGCAA
AGAAAAGAAGGCAGCAAAACTTTTGA

SEQ ID NO: 5

METVQLRNPPRRQLKKLDEDSLTKQPEEVFDVLEKLGEGSYGSVYKAIHK
ETGQIVAIRQVPVESDLQEIIKEISIMQQCDSPHVVKYYGSYFKNTDLWI
VMEYCGAGSVSDIIRLRNKTLTEDEIATILQSTLKGLEYLHFMRKIHRDI
KAGNILLNTEGHAKLADFGVAGQLTDTMAKRNTVIGTPFWMAPEVIQEIG
YNCVADIWSLGITAIEMAEGKPPYADIHPMPAIFMIPTNPPPTFRKPELW
SDNFTDFVKQCLVKSPEQRATATQLLQHPFVRSAKGVSILRDLINEAMDV
KLKRQESQQREVDQDDEENSEEDEMDSGTMVRAVGDEMGTVRVASTMTDG
ANTMIEHDDTLPSQLGTMVINAEDEEEEGTMKRRDETMQPAKPSFLEYFE
QKEKENQINSFGKSVPGPLKNSSDWKIPQDGDYEFLKSWTVEDLQKRLLA
LDPMMEQEIEEIRQKYQSKRQPILDAIEAKKRRQQNF

SEQ ID NO: 6

MEHSVPKNKLKKLSEDSLTKQPEEVFDVLEKLGEGSYGSVFKAIHKESGQ
VVAIKQVPVESDLQEIIKEISIMQQCDSPYVVKYYGSYFKNTDLWIVMEY
CGAGSVSDIIRLRNKTLTEDEIATVLKSTLKGLEYLHFMRKIHRDIKAGN
ILLNTEGHAKLADFGVAGQLTDTMAKRNTVIGTPFWMAPEVIQEIGYNCV
ADIWSLGITSIEMAEGKPPYADIHPMRAIFMIPTNPPPTFRKPEHWSDDF
TDFVKKCLVKNPEQRATATQLLQHPFIVGAKPVSILRDLITEANDMKAKR
QQEQQRELEEDDENSEEEVEVDSHTMVKSGSESAGTMRATGTMSDGAQTM
IEHGSTMLESNLGTMVINSDDEEEEEDLGSMRRNPTSQQIQRPSFMDYFD
KQDSNKAQEGFNHNQQDPCLISKTAFPDNWKVPQDGDFDFLKNLDFEELQ
MRLTALDPMMEREIEELRQRYTAKRQPILDAMDAKKRRQQNF

SEQ ID NO 7

LADFGVAGQLTDTKQIK

SEQ ID NO 8

TLIEDEIATILK

SEQ ID NO 9

AGNILLNTEGHAK

SEQ ID NO 10

ATATQLLQHPFVR

1-29. (canceled)

30. A method for producing a medicament for the treatment of a thromboembolic disorder by combining a Mst protein or a nucleotide sequence encoding a Mst protein with one or more pharmaceutically acceptable carriers or auxiliary substances resulting in a pharmaceutically active formulation.

31. The method according to claim 30, wherein said Mst protein is selected from Mst 1 and Mst 2.

32. The method according to claim 31, wherein said Mst protein is a human Mst protein.

33. The method according to claim 32 wherein the amino acid sequence of said human Mst 1 protein is the amino acid sequence of SEQ ID NO: 1 and the amino acid sequence of said human Mst 2 protein is the amino acid sequence of SEQ ID NO: 3.

34. The method according to claim 32 wherein the nucleotide sequence encoding said human Mst 1 is the nucleotide sequence of SEQ ID NO: 2 and the nucleotide sequence encoding said human Mst 2 is the nucleotide sequence of SEQ ID NO: 4.

35. A method of screening a modulator of a Mst protein or the nucleotide sequence encoding a Mst protein comprising the steps of
i contacting a Mst protein or the nucleotide sequence encoding a Mst protein in an assay selected from the group consisting of a thrombosis-related assay, a kinase assay and a reporter based cell system assay with a test compound and
ii measuring or detecting the activation or inhibition of said test compound on the biological activity of said Mst protein.

36. The method according to claim 35 wherein said thrombosis-related assay is a thrombocyte aggregation assay.

37. The method according to claim 35 further comprising the step of:
iii. selecting said test compound with an activity against a thromboembolic disorder by comparing the changes in said assay in the presence and in the absence of the test compound.

38. The method according to claim 35 wherein said Mst protein is selected from Mst 1 and Mst 2.

39. The method according to claim 38 wherein said Mst protein is a human Mst protein.

40. The method according to claim 39 wherein the amino acid sequence of said human Mst 1 protein is the amino acid sequence of SEQ ID NO: 1 and the amino acid sequence of said human Mst 2 protein is the amino acid sequence of SEQ ID NO: 3.

41. The method according to claim 39, wherein the nucleotide sequence encoding human Mst 1 is the nucleotide sequence of SEQ ID NO: 2 and the nucleotide sequence encoding human Mst 2 is the nucleotide sequence of SEQ ID NO: 4.

42. The method according to claim 35 wherein said test compound is provided in the form of a chemical compound library.

43. The method according to claim 35 wherein the method is carried out on an array.

44. The method according to claim 35 wherein the method is carried out in a robotics system.

45. The method according to claim 35 wherein the method is a method of high-through put screening of the test compound.

46. The method according to claim 35 wherein said test compound detected is an inhibitor of platelet activation or platelet aggregation.

47. The method according to claim 35 wherein said test compound detected reduces the risk for thrombus formation or blood clotting.

48. The method according to claim 30 wherein said thromboembolic disorder is caused by activation or aggregation of platelets.

49. The method according to claim 30 wherein said thromboembolic disorder is selected from myocardial infarction, unstable angina, acute coronary syndromes, coronary artery disease, restenosis, stroke, transient ischemic attacks, pulmonary embolism, left ventricular dysfunction, secondary prevention of clinical vascular complications in patients with cardiovascular and/or cerebrovascular diseases, atherosclerosis and/or comedication to vascular interventions, in particular stroke, myocardial infarction, atherosclerosis and/or restenosis.


SEQUENCE LISTING (10 sequences)

SEQ ID NO: 1 - protein, 487 aa, Homo sapiens
METVQLRNPPRRQLKKLDEDSLTKQPEEVFDVLEKLGEGSYGSVYKAIHKETGQIVAIKQVPVESDLQEIIKEISIMQQCDSPHVVKYYGSYFKNTDLWIVMEYCGAGSVSDIIRLRNKTLTEDEIATILQSTLKGLEYLHFMRKIHRDIKAGNILLNTEGHAKLADFGVAGQLTDTMAKRNTVIGTPFWMAPEVIQEIGYNCVADIWSLGITAIEMAEGKPPYADIHPMRAIFMIPTNPPPTFRKPELWSDNFTDFVKQCLVKSPEQRATATQLLQHPFVRSAKGVSILRDLINEAMDVKLKRQESQQREVDQDDEENSEEDEMDSGTMVRAVGDEMGTVRVASTMTDGANTMIEHDDTLPSQLGTMVINAEDEEEEGTMKRRDETMQPAKPSFLEYFEQKEKENQINSFGKSVPGPLKNSSDWKIPQDGDYEFLKSWTVEDLQKRLLALDPMMEQEIEEIRQKYQSKRQPILDAIEAKKRRQQNF

SEQ ID NO: 2 - DNA, 1464 bp, Homo sapiens
atggagacggtacagctgaggaacccgccgcgccggcagctgaaaaagttggatgaagatagtttaaccaaacaaccagaagaagtatttgatgtcttagagaaacttggagaagggtcctatggcagcgtatacaaagctattcataaagagaccggccagattgttgctattaagcaagttcctgtggaatcagacctccaggagataatcaaagaaatctctataatgcagcaatgtgacagccctcatgtagtcaaatattatggcagttattttaagaacacagacttatggatcgttatggagtactgtggggctggttctgtatctgatatcattcgattacgaaataaaacgttaacagaagatgaaatagctacaatattacaatcaactcttaagggacttgaataccttcattttatgagaaaaatacaccgagatatcaaggcaggaaatattttgctaaatacagaaggacatgcaaaacttgcagattttggggtagcaggtcaacttacagataccatggccaagcggaatacagtgataggaacaccattttggatggctccagaagtgattcaggaaattggatacaactgtgtagcagacatctggtccctgggaataactgccatagaaatggctgaaggaaagcccccttatgctgatatccatccaatgagggcaatcttcatgattcctacaaatcctcctcccacattccgaaaaccagagctatggtcagataactttacagattttgtgaaacagtgtcttgtaaagagccctgagcagagggccacagccactcagctcctgcagcacccatttgtcaggagtgccaaaggagtgtcaatactgcgagacttaattaatgaagccatggatgtgaaactgaaacgccaggaatcccagcagcgggaagtggaccaggacgatgaagaaaactcagaagaggatgaaatggattctggcacgatggttcgagcagtgggtgatgagatgggcactgtccgagtagccagcaccatgactgatggagccaatactatgattgagcacgatgacacgttgccatcacaactgggcaccatggtgatcaatgcagaggatgaggaagaggaaggaactatgaaaagaagggatgagaccatgcagcctgcgaaaccatcctttcttgaatattttgaacaaaaagaaaaggaaaaccagatcaacagctttggcaagagtgtacctggtccactgaaaaattcttcagattggaaaataccacaggatggagactacgagtttcttaagagttggacagtggaggaccttcagaagaggctcttggccctggaccccatgatggagcaggagattgaagagatccggcagaagtaccagtccaagcggcagcccatcctggatgccatagaggctaagaagagacggcaacaaaacttctga

SEQ ID NO: 3 - protein, 491 aa, Homo sapiens
MEQPPAPKSKLKKLSEDSLTKQPEEVFDVLEKLGEGSYGSVFKAIHKESGQVVAIKQVPVESDLQEIIKEISIMQQCDSPYVVKYYGSYFKNTDLWIVMEYCGAGSVSDIIRLRNKTLIEDEIATILKSTLKGLEYLHFMRKIHRDIKAGNILLNTEGHAKLADFGVAGQLTDTMAKRNTVIGTPFWMAPEVIQEIGYNCVADIWSLGITSIEMAEGKPPYADIHPMRAIFMIPTNPPPTFRKPELWSDDFTDFVKKCLVKNPEQRATATQLLQHPFIKNAKPVSILRDLITEAMEIKAKRHEEQQRELEEEEENSDEDELDSHTMVKTSVESVGTMRATSTMSEGAQTMIEHNSTMLESDLGTMVINSEDEEEEDGTMKRNATSPQVQRPSFMDYFDKQDFKNKSHENCNQNMHEPFPMSKNVFPDNWKVPQDGDFDFLKNLSLEELQMRLKALDPMMEREIEELRQRYTAKRQPILDAMDAKKRRQQNF

SEQ ID NO: 4 - DNA, 1476 bp, Homo sapiens
atggagcagccgccggcgcctaagagtaaactaaaaaagctgagtgaagacagtttgactaagcagcctgaagaagtttttgatgtattagagaagcttggagaagggtcttatggaagtgtatttaaagcaatacacaaggaatccggtcaagttgtcgcaattaaacaagtacctgttgaatcagatcttcaggaaataatcaaagaaatttccataatgcagcaatgtgacagcccatatgttgtaaagtactatggcagttattttaagaatacagacctctggattgttatggagtactgtggcgctggctctgtctcagacataattagattacgaaacaagacattaatagaagatgaaattgcaaccattcttaaatctacattgaaaggactagaatatttgcactttatgagaaaaatacacagagatataaaagctggaaatattctcctcaatacagaaggacatgcaaaattggcagattttggagtggctggtcagttaacagatacaatggcaaaacgcaatactgtaataggaactccattttggatggctcctgaggtgattcaagaaataggctataactgtgtggccgacatctggtcccttggcattacttctatagaaatggctgaaggaaaacctccttatgctgatatacatccaatgagggctatttttatgattcccacaaatccaccaccaacattcagaaagccagaactttggtccgatgatttcaccgattttgttaaaaagtgtttggtgaagaatcctgagcagagagctactgcaacacaacttttacagcatccttttatcaagaatgccaaacctgtatcaatattaagagacctgatcacagaagctatggagatcaaagctaaaagacatgacgaacagcaacgagaattggaagaggaagaagaaaattcggatgaagatgagctggattcccacaccatggtgaagactagtgtgggagagtgtggcaccatgcgggccacaagcacgatgagtgaaggggcccagaccatgattgaacataatagcacgatgttggaatccgacttggggaccatggtgataaacagtgaggatgaggaagaagaagatggaactatgaaaagaaatgcaacctcaccacaagtacaaagaccatctttcatggactactttgataagcaagacttcaagaataagagtcacgaaaactgtaatcagaacatgcatgaacccttccctatgtccaaaaacgtttttcctgataactggaaagttcctcaagatggagactttgactttttgaaaaatctaagtttagaagaactacagatgcggttaaaagcactggaccccatgatggaacgggagatagaagaacttcgtcagagatacactgcgaaaagacagcccattctggatgcgatggatgcaaagaaaagaaggcagcaaaacttttga

SEQ ID NO: 5 - protein, 487 aa, Homo sapiens
METVQLRNPPRRQLKKLDEDSLTKQPEEVFDVLEKLGEGSYGSVYKAIHKETGQIVAIRQVPVESDLQEIIKEISIMQQCDSPHVVKYYGSYFKNTDLWIVMEYCGAGSVSDIIRLRNKTLTEDEIATILQSTLKGLEYLHFMRKIHRDIKAGNILLNTEGHAKLADFGVAGQLTDTMAKRNTVIGTPFWMAPEVIQEIGYNCVADIWSLGITAIEMAEGKPPYADIHPMRAIFMIPTNPPPTFRKPELWSDNFTDFVKQCLVKSPEQRATATQLLQHPFVRSAKGVSILRDLINEAMDVKLKRQESQQREVDQDDEENSEEDEMDSGTMVRAVGDEMGTVRVASTMTDGANTMIEHDDTLPSQLGTMVINAEDEEEEGTMKRRDETMQPAKPSFLEYFEQKEKENQINSFGKSVPGPLKNSSDWKIPQDGDYEFLKSWTVEDLQKRLLALDPMMEQEIEEIRQKYQSKRQPILDAIEAKKRRQQNF

SEQ ID NO: 6 - protein, 492 aa, Brachydunio rerio
MEHSVPKNKLKKLSEDSLTKQPEEVFDVLEKLGEGSYGSVFKAIHKESGQVVAIKQVPVESDLQEIIKEISIMQQCDSPYVVKYYGSYFKNTDLWIVMEYCGAGSVSDIIRLRNKTLTEDEIATVLKSTLKGLEYLHFMRKIHRDIKAGNILLNTEGHAKLADFGVAGQLTDTMAKRNTVIGTPFWMAPEVIQEIGYNCVADIWSLGITSIEMAEGKPPYADIHPMRAIFMIPTNPPPTFRKPEHWSDDFTDFVKKCLVKNPEQRATATQLLQHPFIVGAKPVSILRDLITEAMDMKAKRQQEQQRELEEDDENSEEEVEVDSHTMVKSGSESAGTMRATGTMSDGAQTMIEHGSTMLESNLGTMVINSDDEEEEEDLGSMRRNPTSQQIQRPSFMDYFDKQDSNKAQEGFNHNQQDPCLISKTAFPDNWKVPQDGDFDFLKNLDFEELQMRLTALDPMMEREIEELRQRYTAKRQPILDAMDAKKRRQQNF

SEQ ID NO: 7 - protein, 17 aa, Artificial
  Peptides
LADFGVAGQLTDTKQIK

SEQ ID NO: 8 - protein, 12 aa, Artificial
  Peptides
TLIEDEIATILK

SEQ ID NO: 9 - protein, 13 aa, Artificial
  Peptides
AGNILLNTEGHAK

SEQ ID NO: 10 - protein, 13 aa, Artificial
  Peptides
ATATQLLQHPFVR